(19) United States
(12) Patent Application Publication
(10) Pub. No.: US 2014/0276977 A1
(43) Pub. Date: Sep. 18, 2014

(54) Uterine Artery Ligation Devices and Methods

(71) Applicant: RESET MEDICAL, INC., San Clemente, CA (US)

(72) Inventors: Fred Burbank, Laguna Niguel, CA (US); Mike Jones, San Clemente, CA (US); George Vilos, Ontario (CA); Ed Olson, Lake Forest, CA (US)

(73) Assignee: RESET MEDICAL, INC., San Clemente, CA (US)

(21) Appl. No.: 13/835,724

(22) Filed: Mar. 15, 2013

(51) Int. Cl. A61B 17/12

(52) U.S. Cl. CPC A61B 17/12009
USPC 606/144

(57) ABSTRACT

A device useful for ligating an anatomical structure, e.g., a uterine artery, includes a crochet hook-like shaft, and tubular filament holder, and a housing that includes a thumb-slide attached to the shaft. After inserting the filament holder into tissue adjacent to the artery, the shaft is inserted to a point next to the holder, and a hook on the end of the shaft is used to snare the filament and pull it around the artery. The filament can then be tied down, ligating the artery.

### BACKGROUND

1. Field of Endeavor

The present invention relates to devices, systems, and processes useful for restricting the blood flow through a blood vessel, and more specifically to restricting the blood flow through a patient's uterine artery.

2. Brief Description of the Related Art

Burbank et al. have previously described (see, e.g., U.S. Pat. Nos. 6,550,482, 7,223,279, and 7,229,465, among others) beneficial effects that can be produced by temporary uterine hypoxia or anoxia, e.g., by occluding the uterine artery. While devices have therefore been proposed that can be useful for locating and clamping the uterine artery, some have been complicated, expensive, and for other reasons have not been fully embraced.

There remains a need, therefore, for improved devices and methods that can provide simple, economical, and effective access to a female patient's uterine artery.

### SUMMARY

According to a first aspect of the invention, device useful for ligating an anatomical structure comprises an elongate shaft having a proximal end and a distal end, the shaft distal end comprising a distal-facing hook, an elongate tube comprising a proximal end, a distal end, and side port adjacent to said distal end, and a lumen extending proximally from said side port, a housing, wherein the tube is fixed in said housing and extends distally from the housing, wherein the shaft is partially positioned in said housing, and is longitudinally movable relative to the housing between a proximal position, in which said hook is adjacent to said housing, and a distal position, in which said hook is adjacent to said tube side port, and a thumb-slide attached to the shaft and longitudinally movably mounted to the housing.

According to another aspect of the present invention, a method of ligating an anatomical structure in a patient comprises inserting an elongate filament holder distally through tissue of the patient adjacent to the anatomical structure, the filament holder holding a filament, inserting a hook distally through tissue of the patient adjacent to the anatomical structure and adjacent to the filament holder, grabbing the filament from the filament holder with the hook, and pulling the filament proximally

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates a perspective view of an exemplary embodiment of a ligation device in position adjacent to an artery;

FIG. 2 illustrates a longitudinal cross-sectional view, taken at line A-A in FIG. 3, of the device of FIG. 1;

FIG. 3 illustrates a lateral cross-sectional view, taken at line B-B in FIG. 2, of the device of FIG. 1;

FIG. 4 illustrates a perspective, exploded view of the device of FIG. 1; and

FIGS. 5-11 illustrate steps of an exemplary method of ligating an artery, utilizing the device of FIG. 1.

### DETAILED DESCRIPTION OF EXEMPLARY EMBODIMENTS

Referring to the drawing figures, like reference numerals designate identical or corresponding elements throughout the several figures.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10.”

FIG. 1 illustrates a perspective view of an exemplary ligation device 10, positioned adjacent to an anatomical structure A around which it is desired to form a ligature, e.g., to pass a filament (e.g., a suture or the like) around and ligate the structure. While the example of a uterine artery is used herein to describe devices and processes, the processes are not restricted to the ligation of a uterine artery, and other anatomical structures, including other veins and arteries, can be ligated by methods as described herein. Furthermore, while the term “suture” will be used herein as a non-limiting example of a filament, other filaments can be alternatively used; for example, non-resorbable materials, such as wires formed of biocompatible metal or polymers, can also be used.

The ligation device 10 includes an elongate housing 12, a distally extendible shaft 14 which is partially positioned inside the housing, a suture holder 16 that extends distally from the housing, and a slide 18. In general terms, the slide is connected to the shaft 14, and a suture thread or the like (not illustrated) extends from the proximal (in FIG. 1, left) end of the housing, through the device 10, through the holder 16, and is held outside the distal end (in FIG. 1, right) of the holder. By pushing the slide 18 distally, the shaft 14 is pushed distally out of the housing 12, and hooks the suture at the end of the holder 16. The shaft 14 can then be retracted, pulling the suture out (but not completely) of the holding 16, and the suture thus extends around the artery A. The device 10, with the suture thus encircling the artery A, can be retracted proximally, and the suture tied down to ligate the artery.

With reference to FIG. 2, which illustrates a longitudinal cross-sectional view of the device 10, the top surface of the slide 18 optionally includes a depression 20 sized for a human thumb to be located in the depression and manipulate the slide distally and proximally. The shaft 14 includes a proximal end 22, and distal end 24, tapered portion 28 proximal of the distal end, and a proximally-facing hook 26 at the distal end 24; in some respects, the shaft 14 is much like a crochet needle, with the distal hook 26 configured so that it can hook a suture, as described elsewhere herein.

The housing 12 includes, in a portion above and adjacent the shaft 14, at the proximal portions of the taper 28 when the shaft 14 is in its proximal-most position (FIG. 2), proximal depression 32, a distal depression 36, and a longitudinal track 34 extending between the two depressions. A ball or cylinder 30 is positioned in the proximal depression 32, but is sized so that it can move distally in the track 34 to the distal depression 36. As described elsewhere herein, as the shaft 14 is advanced distally (out of housing 12), the shaft 14 dislodges the ball 30 from the proximal depression 32, and carries the ball along the track 34 until it is pushed into the depression 36. During the distal travel of the shaft 14 and ball 30, the ball pushes down on the shaft, causing it to deflect downward. When the ball 30 thus enters the distal depression 36, the shaft 14 is released and snaps back up; the configuration of the shaft 14, track 34, and depressions 32, 36 are mutually designed so that the shaft snaps back up (and the ball enters the distal depression 36), when the hook 26 is under the suture at the end of the holder 16, and thus the hook 26 hooks the suture from the holder 16. Proximal retraction of the shaft 14, carrying the ensnared suture in the hook 26, causes the ball 30 to follow a reverse path.

The proximal end of the device 10 includes a fitting 50 having a lumen 52 through which the proximal end of a length of suture material can extend. A pre-slit or pierced septum 54 is positioned at the distal end of the lumen 52, which leads to an extension tube 64 extends distally from the septum. The proximal end of the housing 12 includes a cavity 60 in which the fitting 50 is partially positioned, with a spring 62 surrounding the extension tube 64, bearing against a distal wall of the cavity 60 and enlarged portions of the fitting 50, as illustrated in the drawing. The extension tube 64 is thus free to move into and out of a lumen 70 in the housing, biases proximally by the spring 62, which lumen 70 leads to the lumen 40 of the holder 16. When a suture is fitted into the holder 16, it is looped around a distal post 44 on the holder 16, through a top side opening 42 at the distal end of the holder, through the lumen 40, proximally into the lumen 70, through the extension tube 64, through the septum 54, and out the proximal end of the lumen 52. The septum 54 holds the suture in place in the lumens 52, 70, 40, while the spring 62 pushes the fitting 50 (with the suture secured through the septum 54) proximally, thus inhibiting or preventing the distal end of the suture from dislodging from the post 44.

FIG. 3 illustrates a lateral cross-sectional view of the device 10, taken at line B-B in FIG. 2. In addition to structures already described herein, FIG. 3 illustrates that the housing 12 is advantageously formed of two lateral half-housings 12A, 12B, which are minor images of each other, and which are joined at their inner surfaces to form the housing 12. A carriage 80 is positioned in a cavity 92 formed in the housing 12, and more particularly between the two half-housings 12A, 12B. The carriage 80 includes a longitudinally extending recess 82, which is advantageously open on the top or bottom (in this embodiment, the bottom), so that the shaft 14 can be securely retained in the recess 82; thus, longitudinal movement of the carriage 80 moves the shaft 14 longitudinally. A pair of rails 84, 86 are formed in the half-housings 12A, 12B, and overlie portions of the carriage 80, capturing the carriage in the cavity 92, while permitting the carriage to slide longitudinally in the cavity. A pair of upstanding posts 88, 90 are formed on the top surface of the carriage 80, and attach to the slide 18, permitting a use to push the slide distally and proximally, and thus advance the shaft 14 distally and proximally. FIG. 4, the perspective, exploded view of the device 10, assists in appreciating how the device 10 could be assembled.

With continued reference to the drawings figures, an exemplary method of ligating a blood vessel of interest, more advantageously, the uterine artery, will be described. In general terms, the uterine artery is ligated through a small, surgically formed opening (e.g., laparotomy) in one of the vaginal formices (posterior, anterior, left and right lateral). While it is advantageous for the procedure to be done under direct visualization by the gynecologist, because of the relative ease and speed, the procedure can also be performed using other techniques, e.g., radiographic techniques. The uterine artery is located within the broad ligament, a tissue bundle located adjacent to the uterus. In very general terms, after access is made to this tissue bundle, including the uterine artery, a suture (e.g., of Vicryl) is place around the tissue bundle (including the uterine artery) and ligated for a therapeutic effect, as previously described by Burbank et al.

FIG. 5 illustrates the device 10 in a starting position or orientation. The suture grasping hook 26 is positioned within the housing 12 of the device 10. The suture S has been loaded within the tip and body of the device, at post 44.

FIG. 6 illustrates that the device 10 is designed for an artery to be positioned within the curved section 42 of the extended tube 16. The artery placement allows for the “crochet hook” 14 to pass over the top of an artery A.

FIG. 7 illustrates that, once the artery is correctly positioned, the user will actuate the shaft 14 forward using the thumb-slide 12 on the device 10. Once the thumb-slide reaches the end of the stroke, the hook will be positioned beneath the looped suture S. The hook will spring toward the suture, because of the action of the ball or cylinder 30, and grab hold of the suture with the hook located at the tip.

FIG. 8 illustrates that, with the suture hooked on the hook 26, the user will pull back (proximally) on the thumb-slide 18, causing the suture to be pulled toward the device 10 as the hook shifts.

FIG. 9 illustrates a close-up of the suture being grasped by the hook 26, which occurs as the thumb-slide 18 is returned to the starting (proximal) position.

FIG. 10 illustrates that, once the thumb-slide 18 returns to the starting position, the suture S will be pulled fully across the width of the artery A.

FIG. 11 illustrates a close-up of the suture orientation after the thumb-slide 18 has been returned to the starting position. Once the filament has been pulled proximally, it will encircle the anatomical structure of interest, e.g., the uterine artery, and the filament can be tied down onto the artery, including to such an extent that it partially or completely occludes the artery. Once the filament has been tied, the device can be retracted proximally from within the tissue surrounding the, e.g., artery.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

1-7. (canceled)

8. A method of ligating an anatomical structure in a patient, the method comprising:
inserting an elongate filament holder distally through tissue of the patient adjacent to the anatomical structure, the filament holder holding a filament;
inserting a hook distally through tissue of the patient adjacent to the anatomical structure and adjacent to the filament holder;
grabbing the filament from the filament holder with the hook; and
pulling the filament proximally.

9. A method according to claim 8, wherein inserting an elongate filament holder comprises inserting a tube having a side port adjacent to a distal end of the tube, with said filament held in the tube at the side port.

10. A method according to claim 8, wherein inserting a hook comprises inserting a hook at the end of an elongate shaft.

11. A method according to claim 8, further comprising:
after said pulling, ligating the anatomical structure with said filament.

12. A method according to claim 8, wherein:
said anatomical structure comprises a uterine artery; and
ligating comprises at least partially occluding said uterine artery.

13. A method according to claim 8, wherein said filament comprises a suture material.
